(19) United States
(12) Patent Application Publication
(10) Pub. No.: US 2011/0230578 A1
(43) Pub. Date: Sep. 22, 2011

(54) FLAME-RETARDANT COMPOSITE FOAM

(75) Inventors: Maxim Peretolchin, Mannheim (DE); Tobias Heinz Steinke, Speyer (DE); Matthias Pfeiffer, Bohl-Iggelheim (DE); Sabine Fuchs, Mannheim (DE); Ingo Bellin, Mannheim (DE); Klaus Hahn, Kirchheim (DE); Günter Scherr, Ludwigshafen (DE); Karl-Heinz Schmitz, Borrstadt (DE)

(73) Assignee: BASF SE, Ludwigshafen (DE)

(21) Appl. No.: 13/050,185

(22) Filed: Mar. 17, 2011

Related U.S. Application Data
(60) Provisional application No. 61/314,598, filed on Mar. 17, 2010.

(51) Int. Cl. C08J 9/35; C08L 61/28; C08L 25/06; C08K 5/51; C08K 3/06; A61K 6/087

(52) U.S. Cl. 521/136

(57) ABSTRACT

A composite foam comprising
A) foamed particles comprising
   A1) from 25 to 95% by weight of styrene polymer(s),
   A2) if appropriate, a blowing agent, and
   A3) if appropriate, one or more additives, and
B) an aminoplastic component comprising
   B1) from 1 to 65% by weight of cured, if appropriate modified and if appropriate foamed aminoplastic resin,
   B2) from 0.1 to 20% by weight of one or more flame-retardant organic phosphorus compounds having from 5 to 80% by weight phosphorus content (based on the respective compound), and/or expandable graphite,
   B3) if appropriate, one or more flame-retardant synergists for component B2),
   B4) if appropriate, a blowing agent, and
   B5) if appropriate, one or more additives,
where—unless otherwise stated—the percentages by weight are based on the entirety of components A1), B1) and B2),
is particularly suitable as insulation material.

The invention relates to a flame-retardant composite foam comprising a styrene polymer and an aminoplastic, to a composition, and to a process for producing the composite foam, and also to its use as insulation material.

EP-A 0 806 451 describes flame-retardant polystyrene foams which comprise—within the polystyrene foam—a combination of phosphorus compounds and sulfur as flame retardant. It is proposed that the sulfur is, if appropriate, encapsulated, and melamine-formaldehyde resins have also been mentioned here as possible capsule material.

Composite foams which comprise a styrene polymer, such as polystyrene, and an aminoplastic, such as melamine-formaldehyde resins or urea-formaldehyde resins, are known by way of example from DE-A 1 479 972, DE-A 23 52 969, and WO 2008/105595. The latter specification describes addition of a silicon-oxide-containing flame retardant to the foam.

GB 2,362,586 and DE-A 199 10 257 disclose addition of phosphorus-containing compounds or expandable graphite as flame retardant to composite foams made of polystyrene and phenol-formaldehyde resins. Said document does not mention aminoplastics.

US-A 2004/0115455 describes composite foams made of polystyrene and of phenol-formaldehyde resins, where these comprise, as flame retardant, phosphorus-containing compounds, expandable graphite, or small proportions of melamine-formaldehyde resins.

Although the known materials themselves have good properties and have particular suitability as insulation materials, there is nevertheless much room for improvements, inter alia in relation to fire-protection performance, insulation capability, and processability. It has been found that addition of phosphorus-containing compounds and/or of expandable graphite to composite foams based on styrene polymers and on aminoplastics gives these foams excellent flame-retardancy properties, without any adverse effect on mechanical properties when comparison is made with the same product without flame retardant.

The invention therefore provides a composite foam comprising

A) foamed particles comprising
A1) from 25 to 95% by weight of styrene polymer(s),
A2) if appropriate, a blowing agent, and
A3) if appropriate, one or more additives, and
   B) an aminoplastic component comprising
B1) from 1 to 65% by weight of cured, if appropriate modified and if appropriate foamed aminoplastic resin,
B2) from 0.1 to 20% by weight of one or more flame-retardant organic phosphorus compounds having from 5 to 80% by weight phosphorus content (based on the respective compound), and/or expandable graphite,
B3) if appropriate, one or more flame-retardant synergists for component B2),
B4) if appropriate, a blowing agent, and
B5) if appropriate, one or more additives,
   where—unless otherwise stated—the percentages by weight are based on the entirety of components A1), B1) and B2).

The invention also provides a composition for producing the composite foam of the invention, comprising

a) expandable, if appropriate prefoamed, pellets comprising
a1) from 25 to 65% by weight of styrene polymer(s),
a2) a blowing agent, and
a3) if appropriate, one or more additives, and
   b) a solution or dispersion of a curable aminoplastic component, comprising
b1) from 1 to 65% by weight of curable, if appropriate modified aminoplastic resin,
b2) from 0.1 to 20% by weight of one or more flame-retardant organic phosphorus compounds having from 5 to 80% by weight phosphorus content (based on the respective phosphorus compound), and/or expandable graphite,
b3) if appropriate, one or more flame-retardant synergists for component b2),
b4) if appropriate, a blowing agent,
b5) if appropriate, one or more additives,
b6) one or more dispersing agents, and
b7) one or more hardener(s),
   where—unless otherwise stated—the percentages by weight are based on the entirety of components a1), b1) and b2).

The invention also provides a process for producing the foam of the invention, comprising the following steps:
i) provision of a solution or dispersion of, if appropriate modified, aminoplastic-resin component b) via homogeneous mixing of components b1), b2), b6), b7), and, if appropriate, b3) to b5),
ii) provision of the expandable particles a),
iii) if appropriate, prefoaming of the particles a),
iv) mixing of components a) and b) to give a mixture comprising from 25 to 95% by weight of component a1), from 1 to 65% by weight of component b1), and from 0.1 to 20% by weight of component b2), based on the entirety of components a1), b1) and b2),
v) curing of component b1),
vi) if appropriate, foaming of component b1), and
vii) if appropriate, completing the foaming of component a),
   where steps v) and/or vi) and/or vii) can be carried out in any desired sequence and can, if appropriate, be carried out simultaneously.

The invention further provides the use of the composite foam of the invention as insulation material, in particular for buildings.

The density of the composite foam of the invention is generally in the range from 5 to 120 kg/m³, preferably from 8 to 60 kg/m³, particularly preferably from 10 to 35 kg/m³.

Component A) of the moldable foam comprises at least one styrene polymer as component A1). It is preferable that component A1) is composed of one or more, preferably one, styrene polymer(s).

In the invention, the expression styrene polymer comprises polymers based on styrene, alpha-methylstyrene, or a mixture of styrene and alpha-methylstyrene; by analogy, this applies to the styrene content in SAN, AMSAN, ABS, ASA, MBS, and MABS (see below). Styrene polymers of the invention are based on at least 50 parts by weight of styrene and/or alpha-methylstyrene monomers.

Styrene polymers preferably used comprise glassclear polystyrene (GPPS), impact-resistant polystyrene (HIPS), anionically polymerized polystyrene or impact-resistant polystyrene (AIPS), styrene-alpha-methylstyrene copolymers, acrylonitrile-butadiene-styrene polymers (ABS), styrene-acrylonitrile copolymers (SAN), acrylonitrile-alpha-methylstyrene copolymers (AMSAN), acrylonitrile-styrene-acrylate (ASA), methyl acrylate-butadiene-styrene (MBS), or methyl methacrylate-acrylonitrile-butadiene-styrene (MABS) polymers, or a mixture of these or with polyphenylene ether (PPE).

In order to improve mechanical properties or resistance to temperature change, the styrene polymers mentioned can be mixed with thermoplastic polymers, such as polyamides (PA), polyolefins, such as polypropylene (PP) or polyethylene (PE), polyacrylates, such as polymethyl methacrylate (PMMA), polycarbonate (PC), polyesters, such as polyethylene terephthalate (PET) or polybutylene terephthalate (PBT), polyether sulfones (PES), polyether ketones, polyether sulfides (PES), or a mixture thereof, in total proportions of at most 30 parts by weight, preferably in the range from 1 to 10 parts by weight, based on 100 parts by weight of polymer melt, with use of compatibilizers, if appropriate. It is also possible to produce mixtures in the ranges of amounts mentioned with, for example, hydrophobically modified or functionalized polymers or oligomers, or rubbers, such as polyacrylates or polydienes, e.g. with styrene-butadiene block copolymers or with biodegradable aliphatic or aliphatic/aromatic copolyesters.

Examples of suitable compatibilizers are maleic anhydride-modified styrene copolymers, polymers containing epoxy groups, or organosilanes.

Particular preference is given to foams of the invention which comprise polystyrene as component A1) and preferably consist thereof, in particular of the following commercially available grades: Neopor®, Styropor®, and Peripor® (all from BASF SE, Ludwigshafen, Germany).

The molar mass M_{w} of expandable styrene polymers (EPS) is preferably in the range from 120 000 to 400 000 g/mol, particularly preferably in the range from 180 000 to 300 000 g/mol, measured by means of gel permeation chromatography to DIN 55672-1 using refractiometric detection (RI) against polystyrene standards. Because of degradation of molar mass during shear and/or exposure to heat, the molar mass of the expandable polystyrene is generally below the molar mass of the polystyrene used by about 10 000 g/mol.

The polymerization reaction in the production process takes place by way of example via bulk polymerization or solution polymerization, or via emulsion polymerization, suspension polymerization, or dispersion polymerization. Suspension polymerization is preferred.

In the suspension polymerization process it is preferable that styrene is the sole monomer used. However, up to 20% of the weight of styrene can have been replaced by other ethylenically unsaturated monomers, e.g. alkylstyrenes, divinylbenzene, acrylonitrile, 1,1-diphenyl ether, or alpha-methylstyrene. Preference is also given to the monomers from which the preferred polymers are obtainable.

Moldable-foam component A) of the invention is generally produced from a polymer melt.

The polymer melt can also receive admixtures of polymer recyclates of the abovementioned thermoplastic polymers, in particular styrene polymers and expandable styrene polymers (EPS), in amounts which do not substantially impair their properties, the amounts generally being at most 50 parts by weight, in particular from 1 to 20 parts by weight, based on 100 parts by weight of styrene-polymer component A).

Other materials that can be added to the polymer melt are conventional additives A3), such as nucleating agents, fillers, plasticizers, soluble and insoluble inorganic and/or organic dyes and pigments, e.g. IR absorbers, such as carbon black, graphite flakes or aluminum powder, together or with spatial separation, e.g. by way of mixers or ancillary extruders. The amounts generally added of the dyes and pigments are in the range from 0.01 to 30 parts by weight, preferably in the range from 1 to 5 parts by weight, based on 100 parts by weight of component a1). In order to achieve homogeneous and microdisperse distribution of the pigments within the styrene polymer it can be advantageous, particularly in the case of polar pigments, to use a dispersing agent, e.g. organosilanes, polymers containing epoxy groups, or maleic anhydride-grafted styrene polymers. Preferred plasticizers are mineral oils and phthalates, and the amounts that can be used of these are from 0.05 to 10 parts by weight, based on 100 parts by weight of component a).

In addition to flame-retardant components B2) and, if appropriate, B3), the location of which is in the aminoplastic layer between the styrene particles, the styrene polymer particles themselves can also comprise one or more, preferably halogen-free, flame-retardant and/or synergistic compounds—preferably the phosphorus compounds mentioned under components B2) and B3) and, if appropriate, synergists thereof. The proportion of these additional flame-retardant compounds, if they are present, is usually from 0.5 to 5 parts by weight (based on 100 parts by weight of a1)).

The location of the additives of component A3) is within the moldable foam A) and they should therefore be distinguished unambiguously from the additives of component B) located within the aminoplastic resin.

When mixing is used to incorporate a blowing agent into the polymer melt, and the material is extruded and pelletized under pressure, foams used as component A) in the invention, where these are made of expandable styrene polymers (EPS foams), can be processed to give expandable pellets (EPS) and, if appropriate, via subsequent prefoaming of the pellets, to give the moldable EPS foam.

The polymer melt comprising blowing agent generally comprises, based on 100 parts by weight of the polymer melt, a total proportion of from 2 to 10 parts by weight, preferably from 3 to 7 parts by weight, of one or more blowing agents homogeneously distributed. Suitable blowing agents are the physical blowing agents usually used in EPS, for example aliphatic hydrocarbons having from 2 to 7 carbon atoms, alcohols, ketones, ethers, or halogenated hydrocarbons. It is preferable to use isobutane, n-butane, isopentane, n-pentane, or isomer mixtures, examples being mixtures of n- and isopentane.

In order to improve foamability, it is possible to introduce finely distributed internal water droplets into the polymer matrix. This can be achieved for example via addition of water to the molten polymer matrix. Addition of the water can take place at a location upstream of, identical with, or downstream of the location of blowing agent feed. Homogeneous distribution of the water can be achieved by means of dynamic or static mixers. A sufficient amount of water is generally from 0 to 2 parts by weight, preferably from 0.05 to 1.5 parts by weight, based on 100 parts by weight of component A).

Foaming of expandable styrene polymers (EPS) having at least 90% of the internal water in the form of internal water droplets with a diameter in the range from 0.5 to 15 μm gives foams with adequate cell number and with homogeneous foam structure.

The amount of blowing agent and water added is selected in such a way that the expansion capability a of the expandable styrene polymers (EPS), defined as bulk density prior to foaming/bulk density after foaming, is at most 125, preferably from 15 to 100.

The bulk density of the expandable styrene polymer pellets (EPS) used as component A) in the invention is generally at most 700 g/l, preferably in the range from 590 to 660 g/l. When fillers are used, bulk densities in the range from 590 to 1200 g/l can occur, depending on the nature and amount of the filler.

Since the blowing agent escapes slowly via diffusion from the finished foam, it is not possible to state an amount of blowing agent for the foam.

Component B):

The composite foam of the invention comprises, in component B), at least one cured, if appropriate modified and if appropriate foamed aminoplastic resin, i.e. a polycondensate which is obtained via reaction of a carboxy compound with a component comprising an amino, imino, or amide group.

Preferred components B1) are condensates of formaldehyde with melamine, with urea, with urethanes, with cyanamide or dicyanamide, with aromatic amines, and/or with sulfonamides.

Particular preference is given to melamine-formaldehyde (MF) condensates, to melamine-urea-formaldehyde (MUF) condensates, and to urea-formaldehyde (UF) condensates, and particular preference is given to MF condensates. Said condensates can have been etherified to some extent or completely with alcohols, preferably C₁-C₄ alcohols, in particular methanol or ethanol. It is preferable to use etherified and non-etherified urea-formaldehyde, melamine-urea-formaldehyde, and melamine-formaldehyde condensates, or a mixture thereof, and in particular etherified and non-etherified melamine-formaldehyde condensates. The, if appropriate modified, aminoplastic resins B1) can by way of example comprise, as further carbonyl compounds alongside formaldehyde, acetaldehyde, trimethylolacetaldehyde, acrolein, furfurol, glyoxal, phthalaldehyde, and/or terephthalaldehyde.

MF, MUF, and UF condensates are available commercially, by way of example from BASF SE, Ludwigshafen, Germany, with trademarks Kauramin® and Kaurit®.

The curable aminoplastic resin is usually used in the form of solution or dispersion, which is cured for the purposes of production of the composite foam of the invention. The amount of the aminoplastic resin generally present in the solution is from 20 to 70% by weight, in particular from 30 to 60% by weight.

The solution/dispersion of the invention can receive additions not only of components b1) to b8) but also of other auxiliaries and additives known to the person skilled in the art, examples being urea, caprolactam, phenyldiglycol, butanediol, and sucrose.

The production of said aminoplastic resins is well-known and is described by way of example in Ullmann's Encyclopedia of Industrial Chemistry, VCH Verlagsgesellschaft, 1985, Vol. A2, pages 115 to 141, and the citations contained therein.

The aminoplastic resins B1) can be unmodified resins, but they can also have been modified. For the purposes of the invention, “modified” means that from 0 to 30 mol % of the amine component (for example of the melamine), preferably up to 20 mol %, particularly preferably up to 10 mol %, with particular preference up to 5 mol %, can have been replaced by other thermoset-formers known per se, preferably phenol and phenol derivatives.

It is preferable that the aminoplastic resin component B1) is composed of unmodified aminoplastic resin(s).

The composite foam of the invention preferably comprises, as component B2), at least one compound of the formula (I) having from 5 to 80% by weight phosphorus content, based on the phosphorus compound,

(X¹)ₛ═PR¹R²R³ (I)

where the definitions of the symbols and indices in the formula (I) are as follows:
R¹ is C₁-C₁₆-alkyl, C₁-C₁₀-hydroxyalkyl, C₂-C₁₆-alkenyl, C₂-C₁₆-alkoxy, C₂-C₁₆-alkenoxy, C₃-C₁₀-cycloalkyl, C₃-C₁₀-cycloalkoxy, C₆-C₁₀-aryl, C₆-C₁₀-aryloxy, C₆-C₁₀-aryl-C₁-C₁₆-alkyl, C₆-C₁₀-aryl-C₁-C₁₆-alkoxy, SR⁹, COR¹⁰, COOR¹¹, CONR¹²R¹³;
R² is C₁-C₁₆-alkyl, C₁-C₁₀-hydroxyalkyl, C₂-C₁₆-alkenyl, C₂-C₁₆-alkoxy, C₂-C₁₆-alkenoxy, C₃-C₁₀-cycloalkyl, C₃-C₁₀-cycloalkoxy, C₆-C₁₀-aryl, C₆-C₁₀-aryl-C₁-C₁₆-alkyl, C₆-C₁₀-aryl-C₁-C₁₆-alkoxy, SR⁹, COR¹⁰, COOR¹¹, CONR¹²R¹³;
R³ is H, SH, SR⁴, OH, OR⁵, or a
   —(Y¹)ₙ—[P(═X²)ᵤR⁶—(Y²)ₙ]ₘ—P(═X³)ₜR⁷R⁸ group;
      or two groups R¹, R², R³ form, together with the phosphorus atom bonded thereto, a ring system;
X¹, X² and X³ are identical or different, being, independently of one another, O or S;
Y¹ and Y² are identical or different, being O or S;
R⁴, R⁵, R⁹, R¹⁰, R¹¹, R¹², and R¹³ are identical or different, being C₁-C₁₂-alkyl, C₃-C₈-cycloalkyl, which may either have no substitution or may have one or more C₁-C₄-alkyl groups as substituents, or being C₂-C₁₂-alkenyl, C₂-C₁₂-alkynyl, C₆-C₁₀-aryl, or C₆-C₁₀-aryl-C₁-C₄-alkyl;
R⁶, R⁷, and R⁸ are identical or different, being, independently of one another, C₁-C₁₆-alkyl, C₂-C₁₆-alkenyl, C₂-C₁₆-alkoxy, C₂-C₁₆-alkenoxy, C₃-C₁₀-cycloalkyl, C₃-C₁₀-cycloalkoxy, C₆-C₁₀-aryl, C₆-C₁₀-aryloxy, C₆-C₁₀-aryl-C₁-C₁₆-alkyl, C₆-C₁₀-aryl-C₁-C₁₆-alkoxy, SR⁹, COR¹⁰, COOR¹¹, CONR¹²R¹³;
n is 0 or 1 if Y¹ and, respectively, Y² is O, and is 1, 2, 3, 4, 5, 6, 7, or 8 if Y¹ and, respectively, Y² is S;
m is an integer from 0 to 100; and
s, t, and u are, independently of one another, 0 or 1.

The definitions of the symbols and indices in formula (I) are preferably as follows:
R¹ is preferably C₁-C₁₆-alkyl, C₁-C₁₀-hydroxyalkyl, C₂-C₁₆-alkenyl, C₁-C₁₆-alkoxy, C₂-C₁₆-alkenoxy, C₃-C₁₀-cycloalkyl, C₃-C₁₀-cycloalkoxy, C₆-C₁₀-aryl, C₆-C₁₀-aryloxy, C₆-C₁₀-aryl-C₁-C₁₆-alkyl, C₆-C₁₀-aryl-C₁-C₁₆-alkoxy.
R² is preferably C₁-C₁₆-alkyl, C₁-C₁₀-hydroxyalkyl, C₂-C₁₆-alkenyl, C₁-C₁₆-alkoxy, C₂-C₁₆-alkenoxy, C₃-C₁₀-cycloalkyl, C₃-C₁₀-cycloalkoxy, C₆-C₁₀-aryl, C₆-C₁₀-aryl-C₁-C₁₆-alkyl, C₆-C₁₀-aryl-C₁-C₁₆-alkoxy.
R³ is preferably H, SH, SR⁴, OH, OR⁵, or a
   —(Y¹)ₙ—[P(═X²)ᵤR⁶—(Y²)ₙ]ₘ—P(═X³)ₜR⁷R⁸ group.
X¹, X² and X³ are preferably identical or different, being, independently of one another, O or S.
Y¹ and Y² are preferably identical or different, being O or S.
R⁴ and R⁵ are preferably identical or different, being C₁-C₁₂-alkyl, C₃-C₈-cycloalkyl, which may either have no substitution or may have one or more C₁-C₄-alkyl groups as substituents, or being C₂-C₁₂-alkenyl, C₂-C₁₂-alkynyl, C₆-C₁₀-aryl, or C₆-C₁₀-aryl-C₁-C₄-alkyl.
R⁶, R⁷, and R⁸ are preferably identical or different, being, independently of one another, C₂-C₁₆-alkyl, C₂-C₁₆-alkenyl, C₂-C₁₆-alkoxy, C₂-C₁₆-alkenoxy, C₃-C₁₀-cycloalkyl, C₃-C₁₀-cycloalkoxy, C₆-C₁₀-aryl, C₆-C₁₀-aryloxy, C₆-C₁₀-aryl-C₁-C₁₆-alkyl, C₆-C₁₀-aryl-C₁-C₁₆-alkoxy, SR⁹, COR¹⁰, COOR¹¹, CONR¹²R¹³.
n is preferably 1 if Y¹ and, respectively, Y² is 0, being 1 or 2 if Y² is S, and
m is preferably an integer from 0 to 10.
s, t, and u are preferably 1.

Preference is given to compounds of the formula (I) in which the definitions of all of the symbols and indices are the preferred definitions.

Preference is also given to compounds of the formula (I) in which two moieties R¹, R², R³ together form a ring system.

It is particularly preferable that the definitions of the symbols and indices in formula (I) are as follows:
R¹ is particularly preferably C₁-C₈-alkyl, C₁-C₈-alkoxy, cyclohexyl, phenyl, phenoxy, benzyl, benzyloxy.
R² is particularly preferably C₁-C₈-alkyl, C₁-C₈-alkoxy, cyclohexyl, phenyl, benzyl, benzyloxy.
R³ is particularly preferably H, SH, SR⁴, OH, OR⁵ or a
   —(Y¹)ₙ—P(═X³)ₜR⁷R⁸ group.
X¹ and X³ are particularly preferably identical or different, being O or S.
Y¹ is particularly preferably O or S.
R⁴ and R⁵ are particularly preferably identical or different, being C₁-C₈-alkyl, cyclohexyl, phenyl, or benzyl.
R⁷ and R⁸ are particularly preferably identical or different, being C₁-C₈-alkyl, C₁-C₈-alkoxy, cyclohexyl, phenyl, phenoxy, benzyl, or benzyloxy.
n is particularly preferably 1 if Y¹ is O, being 1 or 2 if Y¹ is S.
s and t are particularly preferably 1.

Particular preference is given to compounds of the formula (I) in which the definitions of the symbols and indices are the particularly preferred definitions.

It is in particular preferable that the definitions of the symbols and indices in formula (I) are as follows:
R¹ is with particular preference phenyl, phenyloxy.
R² is with particular preference phenyl.
R³ is with particular preference H, SH, SR⁴, OH, OR⁵, or a
   —(Y¹)ₙ—P(═X³)ₜR⁷R⁸ group.
X¹ and X³ are with particular preference identical or different, being O or S.
Y¹ is with particular preference O or S.
R⁴ and R⁵ are with particular preference identical or different, being cyclohexyl, phenyl, or benzyl.
R⁷ and R⁸ are with particular preference identical or different, being phenyl, phenoxy.
n is with particular preference 1 if Y¹ is O, being 1 or 2 if Y¹ is S.
s and t are with particular preference 1.

Preference is in particular given to compounds of the formula (I) in which the definitions of the symbols and indices are the definitions that are in particular preferred.

Preference is in particular further given to the following groups of compounds of the formula (I):

S═PR¹R²—H (Ia)

S═PR¹R²—SH (Ib)

S═PR¹R²—OH (Ic)

S═PR¹R²—S-phenyl (Id)

S═PR¹R²—O-phenyl (Ie)

S═PR¹R²—S-benzyl (If)

S═PR¹R²—O-benzyl (Ig)

S═PR¹R²—P(═S)R⁷R⁸ (Ih)

S═PR¹R²—S—P(═S)R⁷R⁸ (Ii)

S═PR¹R²—S—S—P(═S)R⁷R⁸ (Ij)

S═PR¹R²—O—P(═S)R⁷R⁸ (Ik)

O═PR¹R²—H (Il)

O═PR¹R²—SH (Im)

O═PR¹R²—OH (In)

O═PR¹R²—S-phenyl (Io)

O═PR¹R²—O-phenyl (Ip)

O═PR¹R²—S-benzyl (Iq)

O═PR¹R²—P(═S)R⁷R⁸ (Ir)

O═PR¹R²—S—P(═S)R⁷R⁸ (Is)

O═PR¹R²—S—S—P(═S)R⁷R⁸ (It)

O═PR¹R²—O—P(═S)R⁷R⁸ (Iu)

O═PR¹R²—P(═O)R⁷R⁸ (Iv)

O═PR¹R²—S—P(═O)R⁷R⁸ (Iw)

O═PR¹R²—S—S—P(═O)R⁷R⁸ (Ix)

O═PR¹R²—O—P(═O)R⁷R⁸ (Iy)

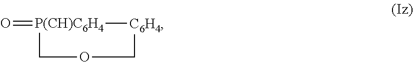

where the definitions of the symbols are as stated in the formula (I).

Preference is in particular given to the following components B2: FSM 1 to FSM 6.

| | | |
|---|---|---|
| 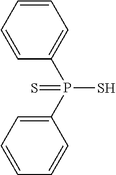 | Diphenyldithiophosphinic acid | FSM1 |
| 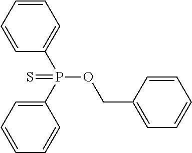 | Benzyl ester of diphenylphosphine sulfide | FSM2 |
| 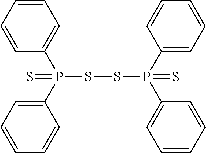 | Bis(diphenylphosphinethioyl) disulfide | FSM3 |
| 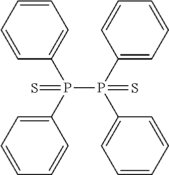 | 1,1,2,2- Tetraphenyldiphosphine disulfide | FSM4 |
| 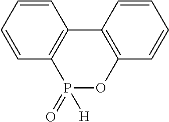 | DOPO | FSM5 |
| 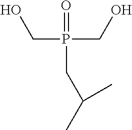 | Bis(hydroxymethyl) isobutylphosphine oxide | FSM6 |

It is preferable that 1 compound of the formula (I) is used as component B2).

Preference is further given to a mixture of two or more, particularly preferably from two to four, in particular two, compounds of the formula (I) as flame retardant.

Some of the compounds of the formula (I) are available commercially, e.g. FSM1 from ABCR GmbH & Co KG, Karlsruhe, Germany, FSM5 in the form of HCA from Sanko, and FSM6 in the form of Cyagard RF-1241 from Cytech.

The flame retardants FMS 2, 3, and 4 can by way of example be produced as in the following references:
FSM2: J. I. G. Cadogan; J. B. Husband; H. McNab; J. Chem. Soc. Perkin Trans. I.; 1983; 1489 to 1495.
FSM3: M. G. Zimin, N. G. Zabirov; V. Smirnov; Zhournal Obschei Khimii; 1980; 50; 1; 24 to 30.
FSM4: W. Kuchen, H. Buchwald, Chem. Ber. 1958, 91, 2871 to 2877.

Preference is further given to expandable graphite as component B2). The layer-lattice structure of graphite allows it to form specific types of intercalation compounds. In these intercalation compounds, foreign atoms or foreign molecules have been absorbed, sometimes in stoichiometric ratios, into the spaces between the carbon atoms. Said graphite compounds, e.g. with sulfuric acid as foreign molecule, are also produced on an industrial scale and are termed expandable graphite. The density of said expandable graphite is in the range from 1.5 to 2.1 g/cm³, and particle size is from 100 to 1000 μm.

On exposure to heat, the thermolysis causes the layers to be separated from one another in the manner of a concertina, and the graphite flakes expand. The expansion can start at temperatures as low as approx. 150° C., where this depends on the type of expandable graphite, and can take place almost instantaneously. In the case of free expansion, the final volume can be many hundred times the initial volume.

The expanded expandable graphite forms an intumescent layer on the surface of the foam and thus slows the spread of fire.

Expandable graphite is available commercially, for example from Graphit Kropfmühl AG, Hauzenberg, Germany.

It is preferable to use expandable graphite alone as component B2).

It is further preferable to use a mixture of one or more of the phosphorus compounds mentioned and expandable graphite.

It is further preferable that component B2) comprises only one or more of the phosphorus compounds mentioned.

The composite foam of the invention comprises, alongside the flame-retardant compounds of component B2), if appropriate, one or more flame retardant synergists. Preference is given to synergists from the group of: elemental sulfur, thermal free-radical generators, such as dicumyl peroxide, di-tert-butyl peroxide, and biscumyl (2,3-diphenyl-2,3-dimethylbutane), metal oxides, metal hydroxides, such as Sb₂O₃, and Sn compounds, and compounds that comprise or liberate nitroxyl radicals.

Expandable graphite is preferably used without synergist B3).

Elemental sulfur is preferred as synergist B3), in particular in combination with one or more of the phosphorus compounds stated, in particular those of the formula (I).

The elemental sulfur preferably has substantially homogeneous distribution within the polymer foam, and this can by way of example be achieved via admixture during the extrusion process or via static or dynamic mixers (e.g. kneaders).

The elemental sulfur can also be used in the form of starting compounds which are decomposed to elemental sulfur under the conditions of the process.

The ratio by weight of component B2) (flame-retardant compound(s)) to component B3) (synergist(s)) is generally 1: from 0.1 to 10, preferably 1: from 0.2 to 7, particularly preferably 1: from 0.3 to 5, and in particular 1: from 0.3 to 3.

If complete freedom from halogen is not necessary, reduced-halogen-content polymer compositions can be produced via the use of component B2) of the invention and the addition of relatively small amounts of halogen-containing, in particular brominated, flame retardants, such as hexabromocyclododecane (HBCD), or brominated styrene homo- or copolymers/oligomers (e.g. styrene-butadiene copolymers as described in WO-A 2007/058736), preferably in amounts in the range from 0.05 to 1 part by weight, in particular from 0.1 to 3.0 parts by weight (based on 100 parts by weight of component B1).

Examples of suitable additives B5) used if appropriate are the above-mentioned (component A3)) fillers, dyes, and pigments, which are added, if appropriate, to component A) and/or to component B).

Inorganic nanoparticles, for example as described in WO 2007/048731, are also suitable as additives B5).

Inorganic nanoparticles used are advantageously aluminum oxide, silicon oxide, titanium dioxide, tin oxide, yttrium oxide, cerium oxide, zinc oxide, and/or silicon carbide. It is preferable to use aluminum oxide, silicon oxide, and/or silicon carbide.

The average diameter of said particles is advantageously from 2 to 500 nm, preferably from 3 to 150 nm, particularly preferably from 5 to 60 nm, in particular from 10 to 40 nm.

Other preferred additives B5) are urea, caprolactam, phenyldiglycol, butanediol, and/or sucrose, and conventional additives such as wetting agents and catalysts.

To produce the composite foam of the invention, the aminoplastic resin b1) is typically used in a mixture with one or more of components b6), b7), and, if appropriate, b4) and b5):

Component b4)—Blowing Agent:

In principle, either physical or chemical blowing agents can be used in the process of the invention (Encyclopedia of Polymer Science and Technology, Vol. I, 3^{rd} edition, Additives chapter, pages 203 to 218, 2003).

“Physical” or “chemical” blowing agents are suitable. “Physical” blowing agents here are volatile liquids or compressed gases which act as blowing agents via physical treatment (e.g. heat or pressure). “Chemical” blowing agents here are those which act as blowing agents via chemical reaction or chemical decomposition with liberation of gas.

Examples of suitable “physical” blowing agents are hydrocarbons, such as pentane, hexane, and halogenated, in particular chlorinated and/or fluorinated, hydrocarbons, such as methylene chloride, chloroform, trichloroethane, fluorochlorocarbons, partially halogenated fluorochlorocarbons (HCFCs), alcohols, such as methanol, ethanol, and n- or isopropanol, ethers, ketones, and esters, such as methyl formate, ethyl formate, methyl acetate, or ethyl acetate, in liquid form, or air, nitrogen, and carbon dioxide in the form of gases.

Examples of suitable “chemical” blowing agents are isocyanates in a mixture with water, where carbon dioxide is liberated as effective blowing agent. Carbonates and bicarbonates in a mixture with acids are also suitable, and likewise generate carbon dioxide. Azo compounds, e.g. azodicarbonamide, are also suitable.

The blowing agent is composed of one or more compounds.

Component b6)—Dispersing Agent:

The dispersing agent or emulsifier used can comprise anionic, cationic, and nonionic surfactants, or else a mixture thereof.

Examples of suitable anionic surfactants are diphenylene oxide sulfonates, alkane- and alkylbenzenesulfonates, alkylnaphthalenesulfonates, olefinsulfonates, alkyl ether sulfonates, fatty alcohol sulfates, ether sulfates, alpha-sulfofatty acid esters, acylaminoalkanesulfonates, acylisothionates, alkyl ether carboxylates, N-acylsarcosinates, alkyl phosphates and alkyl ether phosphates. Nonionic surfactants that can be used comprise, e.g., alkylphenol polyglycol ethers, fatty alcohol polyglycol ethers, fatty acid polyglycol ethers, fatty acid alkanolamides, ethylene oxide/propylene oxide block copolymers, amine oxides, glycerol fatty acid esters, sorbitan esters, and alkyl polyglycosides. Examples of cationic emulsifiers that can be used are alkyltriammonium salts, alkylbenzyldimethylammonium salts, and alkylpyridinium salts.

Component b7)—Hardener:

Hardeners that can be used are in particular acidic compounds, such as inorganic Brønsted acids, e.g. sulfuric acid or phosphoric acid, organic Brønsted acids, such as acetic acid or formic acid, Lewis acids, and also what are known as latent acids, i.e. salts of the acids mentioned with NH₃, with amines, with aminoalcohols, etc.

The composite foams of the invention can be produced by various processes.

Aminoplastic component b) can, if appropriate, be foamed prior to or after mixing with component a), and the foaming here can take place mechanically, via admixture of air or of other gases, or via blowing agent.

The expandable pellets of the styrene polymer a) are, if appropriate prior to mixing with component b), prefoamed to a desired density in a conventional apparatus suitable for producing EPS. Typical bulk densities of the prefoamed pellets here are in the range from 8 to 50 kg/m³.

In one preferred embodiment, the mixing of components a) and b) takes place via coating of the, if appropriate foamed, aminoplastic resin component b) onto the, if appropriate prefoamed or foamed, moldable-foam component a).

In another embodiment, the interstices between the particles a) of the moldable foam are filled by aminoplastic resin component b).

The mixture of components a) and b) is, if appropriate, further foamed, for example mechanically, via admixture of air or of other gases, or via the use of blowing agents, and then resin component b) is cured at room temperature or with use of heat. The heat can by way of example be introduced via microwave radiation or via IR radiation, or use of an oven. In one preferred embodiment, the mixture is compressed during the hardening process. In alternate embodiments, moldable-foam component a) and/or resin component b) are further foamed or, respectively, expanded. This method can produce various forms of the composite foam.

In one preferred embodiment, composite foam sheets are produced in suitable molds. In another preferred embodiment, the composite foam is utilized in bulk for filling cavities in walls or construction materials, in particular construction blocks, such as hollow bricks or Poroton blocks.

In another preferred embodiment, only a small amount of component b) is used, and the preferred B):A) ratio here is <0.1:1. It is preferable here that the, if appropriate prefoamed, pellets of the moldable foam are coated with a mixture of the components, where the pellets are adhesive-bonded via exposure to heat. Additional adhesive bonding then takes place during the fusion of the pellets, in a manner similar to postfoaming during conventional EPS production.

In another preferred embodiment, the fusion is conducted in conventional slab machines for producing EPS slabs. Here, the heat is introduced via steam or heated air.

The composite foam of the invention is particularly suitable as insulation material for the construction industry, i.e. during construction, and during the renovation and repair of buildings, by way of example for thermal insulation and the production of composite materials.

The examples provide further explanation of the invention.

### EXAMPLES

Materials used:

DOPO=9,10-dihydro-9-oxa-10-phosphaphenanthrene 10-oxide
S=elemental sulfur
Pentane: commercially available mixture of n- and isopentane (sec-pentane)

### EPS=Neopor® P 5200 (BASF SE)

Melamine-formaldehyde precondensate: Kauramin® 630 (BASF SE)

### Example 1

### Production of a Composite Foam Made of Expanded Polystyrene (EPS) and of a Melamine-Formaldehyde Condensate (MF) 1:1.5 with 1% by Weight, Based on the Entirety of the Solids Made of EPS and Melamine-Formaldehyde Condensate, of DOPO/S (Completion of Foaming in a Microwave Oven)

75 parts by weight of a spray-dried MF precondensate (molar ratio 1:17) were dissolved in 25 parts by weight of water. The following were admixed with the solution: 15% of formic acid, 2% of a sodium C₁₂/C₁₅-alkylsulfonate, 5% of pentane (based on the MF precondensate), and 1% of DOPO/S (in a ratio by weight of 2.5:1.5), based on the amount of solids of EPS and MF precondensate. The mixture obtained was vigorously mixed and then treated with 66% by weight of prefoamed polystyrene particles with diameter from 4 to 5 mm and with density 17 g/l. The mixture was then foamed to completion in a polypropylene foam mold via irradiation with microwaves at 2.54 GHz. The foam was dried at 100° C. for 4 h. The density of the resultant composite foam was 35 g/l, and its thermal conductivity was 33 mW/m*K, and it complies with the requirements of fire class B2 to DIN 4102.

### Example 2

### Production of a Composite Foam From EPS-MF 1:1.5 with 1% of DOPO/S Under Elevated Pressure in an Oven

75 parts by weight of a spray-dried MF precondensate (molar ratio 1:1.7) were dissolved in 25 parts by weight of water. The following were admixed with the solution: 15% of formic acid, 2% of a sodium C₁₂/C₁₅-alkylsulfonate, 5% of pentane (in each case based on the solids content of the MF precondensate), and 1% of DOPO/S (in a ratio of 2.5:1.5), based on the amount of solids of EPS and MF precondensate. The mixture obtained was vigorously mixed and then treated with 66% by weight of prefoamed polystyrene particles with diameter 5 mm and with density 17 g/l. The mixture was then compressed by a factor of 40% in a foam mold and heated to 100° C. for 3 h. The foam was removed from the foam mold and dried at 100° C. for a further 4 h. The density of the resultant composite foam was 50 g/l, and its thermal conductivity was 32.7 mW/m*K (measured to DIN 13163), and it complies with the requirements of fire class B2 to DIN 4102.

### Example 3

### Production of a Composite Foam Made of EPS-MF 1:1.5 with 1% of DOPO/S Under Atmospheric Pressure in an Oven

75 parts by weight of a spray-dried MF precondensate (molar ratio 1:1.7) were dissolved in 25 parts by weight of water. The following were admixed with the solution: 15% of formic acid, 2% of a sodium C₁₂/C₁₅-alkylsulfonate, 5% of pentane (in each case based on the solids content of the MF precondensate), and 1% of DOPO/S (in a ratio of 2.5:1.5), based on the amount of solids of EPS and MF precondensate. The mixture obtained was vigorously mixed and then treated with 66% by weight of prefoamed polystyrene particles with diameter 5 mm and with density 17 g/l. The mixture was then heated to 80° C. for 45 minutes in a wooden frame structure. The foam was removed from the foam mold and dried at 25° C. for a further 48 h. The density of the resultant composite foam was 50 g/l, and its thermal conductivity was 32.4 mW/m*K, and it complies with the requirements of fire class B2 to DIN 4102.

### Example 4

### Production of a Composite Foam Made of EPS-MF 1:1.5 Using 2% of Expandable Graphite Under Atmospheric Pressure in an Oven

75 parts by weight of a spray-dried MF precondensate (molar ratio 1:1.7) were dissolved in 25 parts by weight of water. The following were admixed with the solution: 15% of formic acid, 2% of a sodium C₁₂/C₁₋₅-alkylsulfonate, 5% of pentane (in each case based on the solids content of the MF precondensate), and 2% expandable graphite ES 350 F5 from Graphit Kropfmühl AG, based on the amount of solids of EPS and MF precondensate.

The mixture obtained was vigorously mixed and then treated with 66% by weight of prefoamed polystyrene particles with diameter 5 mm and with density 17 g/l. The mixture was then heated to 80° C. for 45 minutes in a wooden frame structure. The foam was removed from the foam mold and dried at 25° C. for a further 48 h. The density of the resultant composite foam was 48 g/l, and its thermal conductivity was 33 mW/m*K, and it complies with the requirements of fire class B2 to DIN 4102.

### Example 5 (Application Example)

### Production of a Construction Element Via Filling of Poroton Block with a Mixture Made of EPS-MF and DOPO/S

A Poroton block (POROTON T-8 without filling, Wienerberger AG, Vienna, Austria) was placed on a sheet of metal and filled with the mixture described in example 3 in such a way as to fill all of the cavities (total volume about 13.2 l). The block was covered with a second sheet of metal, placed in an oven for 45 min at 80° C., and then dried at room temperature for two days. The finished block exhibited secure and stable filling securely located within the cavities; this could be removed by scratching only if large forces were applied.

The entire amount of the filling after 34 days amounted to 615 g (corresponding to an approximate density of 47 kg/m³ for the filling). The filling exhibited excellent flame retardancy, and a gas burner operated with butane with an approximate temperature of 1200° C. caused only slight damage to the filling (less than 2 cm in depth).

1-21. (canceled)

22. A composite foam comprising
A) foamed particles comprising
A1) from 25 to 95% by weight of styrene polymer(s),
A2) optionally a blowing agent, and
A3) optionally one or more additives, and
B) an aminoplastic component comprising
B1) from 1 to 65% by weight of cured, optionally modified and optionally foamed aminoplastic resin,
B2) from 0.1 to 20% by weight of one or more flame-retardant organic phosphorus compounds having from 5 to 80% by weight phosphorus content (based on the respective compound), and/or expandable graphite,
B3) optionally one or more flame-retardant synergists for component B2),
B4) optionally a blowing agent, and
B5) optionally one or more additives,
where—unless otherwise stated—the percentages by weight are based on the entirety of components A1), B1) and B2).

23. The composite foam according to claim 22, where component A1) is polystyrene.

24. The composite foam according to claim 22, where component B1) is an unmodified or modified melamine-formaldehyde condensate, melamine-urea-formaldehyde condensate, or urea-formaldehyde condensate.

25. The composite foam according to claim 22, where component B2) is expandable graphite.

26. The composite foam according to claim 22, where component B2) comprises a phosphorus compound of the formula (I),
(X¹)ₛ═PR¹R²R³ (I)
where the definitions of the symbols and indices in formula (I) are as follows:
R¹ is C₁-C₁₆-alkyl, C₁-C₁₀-hydroxyalkyl, C₂-C₁₆-alkenyl, C₁-C₁₆-alkoxy, C₂-C₁₆-alkenoxy, C₃-C₁₀-cycloalkyl, C₃-C₁₀-cycloalkoxy, C₆-C₁₀-aryl, C₆-C₁₀-aryloxy, C₆-C₁₀-aryl-C₁-C₁₆-alkyl, C₆-C₁₀-aryl-C₁-C₁₆-alkoxy, SR⁹, COR¹⁰, COOR¹¹, CONR¹²R¹³;
R² is C₁-C₁₆-alkyl, C₁-C₁₀-hydroxyalkyl, C₂-C₁₆-alkenyl, C₁-C₁₆-alkoxy, C₂-C₁₆-alkenoxy, C₃-C₁₀-cycloalkyl, C₃-C₁₀-cycloalkoxy, C₆-C₁₀-aryl, C₆-C₁₀-aryl-C₁-C₁₆-alkyl, C₆-C₁₀-aryl-C₁-C₁₆-alkoxy, SR⁹, COR¹⁰, COOR¹¹, CONR¹²R¹³;
R³ is H, SH, SR⁴, OH, OR⁵, or a
—(Y¹)ₙ—[P(═X²)ᵤR⁶—(Y²)ₙ]ₘ—P(═X³)ₜR⁷R⁸ group;
or two groups R¹, R², R³ form, together with the phosphorus atom bonded thereto, a ring system;
X¹, X² and X³ are identical or different, being, independently of one another, O or S;
Y¹ and Y² are identical or different, being O or S;
R⁴, R⁵, R⁹, R¹⁰, R¹¹, R¹², and R¹³ are identical or different, being C₁-C₁₂-alkyl, C₃-C₈-cycloalkyl, which may either have no substitution or may have one or more C₁-C₄-alkyl groups as substituents, or being C₂-C₁₂-alkenyl, C₂-C₁₂-alkynyl, C₆-C₁₀-aryl, or C₆-C₁₀-aryl-C₁-C₄-alkyl;
R⁶, R⁷, and R⁸ are identical or different, being, independently of one another, C₁-C₁₆-alkyl, C₂-C₁₆-alkenyl, C₁-C₁₆-alkoxy, C₂-C₁₆-alkenoxy, C₃-C₁₀-cycloalkyl, C₃-C₁₀-cycloalkoxy, C₆-C₁₀-aryl, C₆-C₁₀-aryloxy, C₆-C₁₀-aryl-C₆-C₁₆-aryl-C₁-C₁₆-alkoxy, SR⁹, COR¹⁰, COOR¹¹, CONR¹²R¹³;
n is 0 or 1 if Y¹ and, respectively, Y² is 0, and is 1, 2, 3, 4, 5, 6, 7, or 8 if Y¹ and, respectively, Y² is S;
m is an integer from 0 to 100; and
s, t, and u are, independently of one another, 0 or 1.

27. The composite foam according to claim 26, wherein the phosphorus compound has been selected from the group consisting of: diphenyldithiophosphinic acid, benzyl ester of diphenylphosphine sulfide, bis(diphenylphosphinethioyl) disulfide, 1,1,2,2-tetraphenyldiphosphine disulfide, bis(hydroxymethyl)isobutylphosphine oxide and

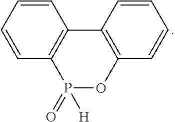

28. The composite foam according to claim 26, comprising elemental sulfur as flame-retardant synergists B3).

29. The composite foam according to claim 22, wherein the aminoplastic resin B1) has not been modified.

30. The composite foam according to claim 22, wherein the aminoplastic component has been foamed.

31. The composite foam according to claim 22, wherein the aminoplastic component has not been foamed.

32. The composite foam according to claim 22, wherein the ratio by weight of components B):A) is >1.

33. The composite foam according to claim 22, wherein the ratio by weight of components B):A) is ≦0.1.

34. A composition for producing the composite foam of the invention according to claim 22, comprising
a) expandable, optionally prefoamed, pellets comprising
a1) from 25 to 65% by weight of styrene polymer(s),
a2) a blowing agent, and
a3) optionally one or more additives, and
b) a solution or dispersion of a curable aminoplastic component, comprising
b1) from 1 to 65% by weight of curable, optionally modified, aminoplastic resin,
b2) from 0.1 to 20% by weight of one or more flame-retardant organic phosphorus compounds having from 5 to 80% by weight phosphorus content (based on the respective phosphorus compound), and/or expandable graphite,
b3) optionally one or more flame-retardant synergists for component b2),
b4) optionally a blowing agent,
b5) optionally one or more additives,
b6) one or more dispersing agents, and
b7) one or more hardener(s),
where—unless otherwise stated—the percentages by weight are based on the entirety of components a1), b1) and b2).

35. The composition according to claim 34, wherein the blowing agent a2) is an aliphatic hydrocarbon which has from 2 to 7 carbon atoms, an alcohol, a ketone, an ether, a halogenated hydrocarbon, or a mixture of said compounds.

36. The composition according to claim 24, wherein component b7) (hardener) is an inorganic or organic Brønsted acid, a Lewis acid, or a latent acid.

37. The composition according to claim 24, wherein component b6) (dispersing agent) has been selected from the group consisting of
a. diphenylene oxide sulfonate,
b. alkane- and alkylbenzenesulfonate,
c. alkylnaphthalenesulfonate,
d. olefinsulfonate,
e. alkyl ether sulfonate,
f. fatty alcohol sulfate,
g. ether sulfate,
h. alpha-sulfofatty acid ester,
i. acylaminoalkanesulfonate,
j. acylisothionate,
k. alkyl ether carboxylate,
l. N-acylsarcosinate,
m. alkyl phosphate and alkyl ether phosphate,
n. alkylphenol polyglycol ether,
o. fatty alcohol polyglycol ether,
p. fatty acid polyglycol ether,
q. fatty acid alkanolamide,
r. ethylene oxide/propylene oxide block copolymers,
s. amine oxide,
t. glycerol fatty acid ester,
u. sorbitan ester,
v. alkyl polyglycoside,
w. alkyltriammonium salt,
x. alkylbenzyldimethylammonium salt, and
y. alkylpyridinium salt.

38. A process for producing a flame-retardant composite foam comprising
A) foamed particles comprising
A1) from 25 to 95% by weight of styrene polymer(s),
A2) optionally a blowing agent, and
A3) optionally one or more additives, and
B) an aminoplastic component comprising
B1) from 1 to 65% by weight of cured, optionally modified and optionally foamed aminoplastic resin,
B2) from 0.1 to 20% by weight of one or more flame-retardant organic phosphorus compounds having from 5 to 80% by weight phosphorus content (based on the respective compound), and/or expandable graphite,
B3) optionally one or more flame-retardant synergists for component B2),
B4) optionally a blowing agent, and
B5) optionally one or more additives,
comprising the following steps:
i) providing a solution or dispersion of aminoplastic-resin component b) via homogeneous mixing of components b1), b2), b6), b7), and optionally b3) to b5),
ii) providing the expandable particles a),
iii) optionally prefoaming of the particles a),
iv) mixing of components a) and b) to give a mixture comprising from 25 to 95% by weight of component a1), from 1 to 65% by weight of component b1), and from 0.1 to 20% by weight of component b2), based on the entirety of components a1), b1) and b2),
v) curing of component b1),
vi) optionally foaming of component b1), and
vii) optionally completing the foaming of component a),
where steps v) and/or vi) and/or vii) can be carried out in any desired sequence and can, optionally be carried out simultaneously and wherein components a), b), and b1) to b7) are as follows
a) expandable, optionally prefoamed, pellets comprising
a1) from 25 to 65% by weight of styrene polymer(s),
a2) a blowing agent, and
a3) optionally one or more additives, and
b) a solution or dispersion of a curable aminoplastic component, comprising
b1) from 1 to 65% by weight of curable, optionally modified, aminoplastic resin,
b2) from 0.1 to 20% by weight of one or more flame-retardant organic phosphorus compounds having from 5 to 80% by weight phosphorus content (based on the respective phosphorus compound), and/or expandable graphite,
b3) optionally one or more flame-retardant synergists for component b2),
b4) optionally a blowing agent,
b5) optionally one or more additives,
b6) one or more dispersing agents, and
b7) one or more hardener(s),
where—unless otherwise stated—the percentages by weight are based on the entirety of components a1), b1) and b2.

39. An insulation material comprising the composite foam according to claim 22.

40. A filling material for cavities comprising the composite foam according to claim 22.

41. A porous construction block filled with the composite foam according to claim 22.

42. The insulation material according to claim 39, wherein the composite foam is in the form of a sheet.
